(19) United States
(12) Patent Application Publication
(10) Pub. No.: US 2017/0106327 A1
(43) Pub. Date: Apr. 20, 2017

(54) PROCESS FOR RECOVERING METHANE FROM A GAS STREAM COMPRISING METHANE AND ETHYLENE

(71) Applicant: SHELL OIL COMPANY, Houston, TX (US)

(72) Inventors: Sivakumar SADASIVAN VIJAYAKUMARI, Gonzales, LA (US); Charles-Edouard SANDERS, Gyungnam (KR)

(21) Appl. No.: 15/311,872

(22) PCT Filed: May 18, 2015
(86) PCT No.: PCT/EP2015/060840

§ 371 Date: Nov. 17, 2016

(30) Foreign Application Priority Data
May 19, 2014 (EP) 14168769.9

(51) Int. Cl. B01D 53/047; B01D 3/14; C07C 7/04; C07C 7/00; C07C 7/11; C07C 7/12

(52) U.S. Cl. CPC B01D 53/047; C07C 7/11; C07C 7/12; C07C 7/04; C07C 7/005; B01D 3/14; B01D 2256/245; B01D 2257/7022

(57) ABSTRACT

The invention relates to a process for recovering methane from a gas stream comprising methane and ethylene, comprising: a sorption step which comprises contacting the gas stream comprising methane and ethylene with a sorption agent which has a lower affinity for methane than for ethylene, resulting in sorption of ethylene and part of the methane by the sorption agent and in a gas stream comprising methane; a rinse step which comprises contacting a gas stream, comprising a compound for which the sorption agent has a higher affinity than for methane, with the sorption agent containing sorbed ethylene and methane, resulting in sorption of the compound for which the sorption agent has a higher affinity than for methane by the sorption agent, in desorption of methane from the sorption agent and in a gas stream comprising methane; and a desorption step which comprises desorbing sorbed ethylene and the sorbed compound for which the sorption agent has a higher affinity than for methane resulting in a gas stream comprising ethylene and the compound for which the sorption agent has a higher affinity than for methane.

### FIELD OF THE INVENTION

The present invention relates to a process for recovering methane from a gas stream comprising methane and ethylene.

### BACKGROUND OF THE INVENTION

It is known to convert methane into saturated and unsaturated, non-aromatic hydrocarbons having 2 or more carbon atoms, including ethylene, by means of a process called “Oxidative Coupling of Methane” (OCM). In this process, a gas stream comprising methane is contacted with an OCM catalyst and with an oxidant, such as oxygen or air. In such a process, two methane molecules are first coupled into one ethane molecule, which is then dehydrogenated into ethylene. Said ethane and ethylene may further react into saturated and unsaturated hydrocarbons having 3 or more carbon atoms, including propane, propylene, butane, butene, etc. Therefore, usually, the gas stream leaving an OCM process contains water, hydrogen, carbon monoxide, carbon dioxide, methane, ethane, ethylene, propane, propylene, butane, butene and saturated and unsaturated hydrocarbons having 5 or more carbon atoms.

In general, the conversion that can be achieved in an OCM process is relatively low. Besides, at a higher conversion, the selectivity decreases so that it is generally desired to keep the conversion low. As a result, a relatively large amount of unconverted methane leaves the OCM process. The proportion of unconverted methane in the OCM product gas stream may be as high as 70 to 80 mol % based on the total molar amount of the gas stream. This unconverted methane has to be recovered from the desired products, such as ethylene and other saturated and unsaturated hydrocarbons having 2 or more carbon atoms, which are also present in such gas streams.

It is known to separate the gas stream leaving an OCM process in the following way. Acid gas (mainly CO₂) is removed in two stages, the first stage is an aqueous monoethanolamine (MEA) absorption system, and the second stage removes final traces of CO₂ by scrubbing against aqueous NaOH. The CO₂-free gas is dried in a dessicant bed and processed in a separation train similar to that used in conventional ethylene plants. The separation sequence comprises a front end demethanizer, deethanizer, C2 splitter, depropanizer, C3 splitter, and a debutanizer. The cryogenic needs for separation are met by a propylene-ethylene cascade refrigeration system that requires ethylene refrigerant only for the demethanization stage.

Thus, it is known to separate methane from saturated and unsaturated hydrocarbons having 2 or more carbon atoms, such as ethylene, by means of cryogenic distillation in so-called “demethanizer” columns. In cryogenic distillation, a relatively high pressure (in general: 23 to 35 bar) and a relatively low (cryogenic) temperature (in general: −120 to −70° C.) are applied to effect the separation of methane. The use of cryogenic distillation following an OCM process is for example disclosed in U.S. Pat. No.5,113,032 and U.S. Pat. No.5,025,108.

An object of the invention is to provide a technically advantageous, efficient and affordable process for recovering methane from a gas stream comprising methane and ethylene, more especially in a case where such gas stream comprises a relatively high proportion of unconverted methane. Such technically advantageous process would preferably result in a lower energy demand and/or lower capital expenditure.

### SUMMARY OF THE INVENTION

Surprisingly it was found that such technically advantageous process, resulting in a lower energy demand and/or lower capital expenditure, for recovering methane from a gas stream comprising methane and ethylene may be provided by subjecting such gas stream to the following three steps:

a sorption step which comprises contacting the gas stream comprising methane and ethylene with a sorption agent which has a lower affinity for methane than for ethylene, resulting in sorption of ethylene and part of the methane by the sorption agent and in a gas stream comprising methane;

a rinse step which comprises contacting a gas stream, comprising a compound for which the sorption agent has a higher affinity than for methane, with the sorption agent containing sorbed ethylene and methane, resulting in sorption of the compound for which the sorption agent has a higher affinity than for methane by the sorption agent, in desorption of methane from the sorption agent and in a gas stream comprising methane; and

a desorption step which comprises desorbing sorbed ethylene and the sorbed compound for which the sorption agent has a higher affinity than for methane resulting in a gas stream comprising ethylene and the compound for which the sorption agent has a higher affinity than for methane.

Accordingly, the present invention relates to a process for recovering methane from a gas stream comprising methane and ethylene, comprising the above-mentioned sorption step, rinse step and desorption step.

### BRIEF DESCRIPTION OF THE DRAWINGS

FIG. 1 shows an embodiment of the process of the present invention, comprising the above-mentioned sorption step, rinse step and desorption step.

FIG. 2 shows an embodiment of the embodiment shown in FIG. 1, in which the gas stream that is subjected to the sorption step additionally comprises components other than methane and ethylene, namely hydrogen, carbon monoxide, carbon dioxide, ethane and hydrocarbons having 3 or more carbon atoms.

### DETAILED DESCRIPTION OF THE INVENTION

The gas stream that is subjected to the sorption step of the process of the present invention is a gas stream which comprises methane and ethylene. Preferably, said gas stream originates from the above-mentioned process of oxidative coupling of methane (OCM), wherein a gas stream comprising methane is contacted with an OCM catalyst and with an oxidant, such as oxygen or air, in order to convert the methane into ethylene and optionally ethane and/or saturated and unsaturated, non-aromatic hydrocarbons having 3 or more carbon atoms. Preferably, the gas stream that is subjected to the sorption step comprises 50 to 99 mol % of methane and 1 to 50 mol % of ethylene. Said relative amounts are based on the total amount of the gas stream.

Within the present specification, where reference is made to relative (e.g. molar) amounts of components in a gas stream, such relative amounts are to be selected such that the total amount of said gas stream does not exceed 100%.

In the sorption step of the process of the present invention, a gas stream comprising methane and ethylene is contacted with a sorption agent which has a lower affinity for methane than for ethylene, resulting in sorption of ethylene and part of the methane by the sorption agent and in a gas stream comprising methane. That is to say, the gas stream resulting from the sorption step comprises methane that is not sorbed by the sorption agent. In particular, the amount of methane in the gas stream resulting from the sorption step is 10 to 99%, preferably 30 to 99%, more preferably 40 to 99%, more preferably 50 to 99%, most preferably 60 to 99%, based on the amount of methane in the gas stream that is subjected to the sorption step. The latter percentage may also be referred to as “methane rejection” (methane not being sorbed, but “rejected”). Such “methane rejection” may be varied by varying the pressure and/or the nature of the sorption agent. Consequently, the amount of methane that is sorbed by the sorption agent in the sorption step is 1 to 90%, preferably 1 to 70%, more preferably 1 to 60%, more preferably 1 to 50%, most preferably 1 to 40%, based on the amount of methane in the gas stream that is subjected to the sorption step.

The amount of methane in the gas stream resulting from the sorption step may be at most 99%, or at most 98%, or at most 95%, or at most 90%, based on the amount of methane in the gas stream that is subjected to the sorption step. Further, the amount of methane in the gas stream resulting from the sorption step may be at least 10%, or at least 30%, or at least 40%, or at least 50%, or at least 60%, based on the amount of methane in the gas stream that is subjected to the sorption step. Thus, said amount of methane in the gas stream resulting from the sorption step may for example be 10 to 99% or 60 to 99%, or 10 to 90% or 60 to 90%. Consequentially, the amount of methane that is sorbed by the sorption agent in the sorption step may be at least 1%, or at least 2%, or at least 5%, or at least 10%, based on the amount of methane in the gas stream that is subjected to the sorption step. Further, the amount of methane that is sorbed by the sorption agent in the sorption step may be at most 90%, or at most 70%, or at most 60%, or at most 50%, or at most 40%, based on the amount of methane in the gas stream that is subjected to the sorption step. Thus, said amount of methane that is sorbed by the sorption agent in the sorption step may for example be 1 to 90% or 1 to 40%, or 10 to 90% or 10 to 40%.

In the sorption step of the process of the present invention, a sorption agent is used. In the present specification, “sorption” means a process in which one substance (the sorption agent) takes up or holds or retains another substance by absorption, adsorption or a combination of both.

Further, said sorption agent used in the sorption step of the process of the present invention has a lower affinity for methane than for ethylene. This means that under the conditions applied in said sorption step, including pressure and temperature which are further defined hereinbelow, said sorption agent has a lower affinity for methane than for ethylene. This implies that in the process of the present invention such sorption agent should be used in the sorption step, that the molar ratio of sorbed ethylene to sorbed methane is greater than 1:1, assuming equal partial pressures for ethylene and methane. Preferably, said ratio is in the range of from 1.1:1 to 10:1, more preferably 1.1:1 to 5:1. Sorption agents suitable to be used in the present invention may be selected by comparing the extent of sorption of methane with the extent of sorption of ethylene under any given temperature and pressure conditions for a variety of known sorption agents, assuming equal partial pressures for ethylene and methane. Therefore, a wide range of sorption agents may be used since the only criterion in the present invention is that the sorption agent should have a lower affinity for methane than for ethylene. Without any limitation, examples of suitable sorption agents are activated carbon, zeolite 13X and zeolite 5A.

The pressure in the sorption step of the process of the present invention may vary within wide ranges. Preferably, said pressure is higher than atmospheric pressure. More preferably, said pressure is higher than atmospheric pressure and at most 15 bar, more preferably of from 5 to 15 bar, most preferably 7 to 13 bar.

The temperature in the sorption step of the process of the present invention may also vary within wide ranges. Preferably, said temperature is in the range of from 0 to 100° C., more preferably 10 to 80° C., most preferably 25 to 50° C. Advantageously, in the present invention, said sorption step may be carried out at a non-cryogenic temperature (e.g. of from 0 to 100° C. as mentioned above).

In the rinse step of the process of the present invention, the sorption agent containing sorbed ethylene and methane resulting from the above-described sorption step is contacted with a gas stream comprising a compound for which the sorption agent has a higher affinity than for methane. Since the sorption agent has a lower affinity for methane than for said compound that is present in the gas stream that is used in the rinse step, said rinse step results in sorption of said compound for which the sorption agent has a higher affinity than for methane by the sorption agent, in desorption of methane from the sorption agent and in a gas stream comprising methane. That is to say, the gas stream resulting from the rinse step comprises methane that is desorbed from the sorption agent.

The gas stream that is used in the rinse step comprises a compound for which the sorption agent has a higher affinity than for methane. Since, in the process of the present invention, the sorption agent has a lower affinity for methane than for ethylene, the gas stream, comprising a compound for which the sorption agent has a higher affinity than for methane, that is used in the rinse step may comprise ethylene. Preferably, said gas stream that is used in the rinse step contains substantially no methane. Generally, said compound for which the sorption agent has a higher affinity than for methane comprises one or more compounds selected from the group consisting of ethylene, ethane and hydrocarbons having 3 or more carbon atoms. Suitably, said hydrocarbons having 3 or more carbon atoms comprise saturated and unsaturated hydrocarbons having 3 or more carbon atoms, including propane, propylene, butane and butene, and optionally saturated and unsaturated hydrocarbons having 5 or more carbon atoms. Preferably, the gas stream comprising a compound for which the sorption agent has a higher affinity than for methane comprises ethylene, ethane or a mixture of ethylene and ethane, more preferably ethylene.

Thus, in the process of the present invention, the sorption agent has a lower affinity for methane than for the compound present in the gas stream that is used in the rinse step. As already discussed above in relation to the sorption step, this means that under the conditions applied in the rinse step, including pressure and temperature which are further defined hereinbelow, said sorption agent has a lower affinity for methane than for said other compound present in the gas stream that is used in the rinse step.

Preferably, the gas stream to be used during the above-mentioned rinse step is a gas stream that is split off from the gas stream comprising ethylene and the compound for which the sorption agent has a higher affinity than for methane which results from the desorption step. In that way, advantageously, no external gas stream would be needed to perform the rinse step and a greater process efficiency and integration is thereby achieved. For example, as discussed above, the gas stream comprising a compound for which the sorption agent has a higher affinity than for methane to be used in the rinse step may comprise ethylene, ethane or a mixture of ethylene and ethane, in which case the gas stream which results from the desorption step comprises ethylene or a mixture of ethylene and ethane.

The pressure in the rinse step of the process of the present invention may vary within wide ranges. Preferably, said pressure is higher than atmospheric pressure. More preferably, said pressure is higher than atmospheric pressure and at most 15 bar, more preferably of from 5 to 15 bar, most preferably 7 to 13 bar.

The temperature in the rinse step of the process of the present invention may also vary within wide ranges. Preferably, said temperature is in the range of from 0 to 100° C., more preferably 10 to 80° C., most preferably 25 to 50° C. Advantageously, in the present invention, said rinse step may be carried out at a non-cryogenic temperature (e.g. of from 0 to 100° C. as mentioned above).

In the desorption step of the process of the present invention, ethylene and the compound for which the sorption agent has a higher affinity than for methane that are sorbed by the sorption agent are desorbed, resulting in a gas stream comprising ethylene and the compound for which the sorption agent has a higher affinity than for methane. That is to say, the latter gas stream resulting from the desorption step comprises ethylene and the compound for which the sorption agent has a higher affinity than for methane that are desorbed from the sorption agent. For example, as discussed above, the gas stream comprising a compound for which the sorption agent has a higher affinity than for methane to be used in the rinse step may comprise ethylene, ethane or a mixture of ethylene and ethane, in which case the gas stream which results from the desorption step comprises ethylene or a mixture of ethylene and ethane.

Preferably, in the desorption step of the process of the present invention, desorption is effected by reducing the pressure. That is to say, the pressure in the desorption step is lower than the pressure in the sorption and rinse steps. This is usually referred to as “Pressure Swing Adsorption” (PSA). In the embodiment wherein desorption in the desorption step is effected by reducing the pressure, the pressure in the sorption and rinse steps is preferably in the range of from 5 to 15 bar, more preferably 7 to 13 bar.

In a case wherein such relatively low pressure (e.g. at most 15 bar) is used in the sorption and rinse steps, advantageously only part of the methane is sorbed in addition to ethylene (in the sorption step) or no methane remains sorbed (in the rinse step). Thus, advantageously, in the sorption and rinse steps of the process of the present invention, a relatively low pressure is applied (e.g. of from 5 to 15 bar as mentioned above). In addition, such low pressure advantageously results in that relatively less compression of the gas stream may be needed. It is especially advantageous that the pressure that may be needed in the sorption step of the process of the present invention may be the same as the pressure in the process from which the gas stream comprising methane and ethylene may originate, such as the pressure in the above-mentioned process of oxidative coupling of methane (OCM). In the latter case, there would be no need at all for any compression of said gas stream in order to carry out said sorption step.

Further, in the embodiment wherein desorption in the desorption step is effected by reducing the pressure, the pressure in the desorption step is preferably in the range of from 0.1 to 3 bar, more preferably 0.5 to 2 bar.

The temperature in the desorption step of the process of the present invention may also vary within wide ranges. Preferably, said temperature is in the range of from 0 to 100° C., more preferably 10 to 80° C., most preferably 25 to 50° C. Advantageously, in the present invention, said desorption step may be carried out at a non-cryogenic temperature (e.g. of from 0 to 100° C. as mentioned above).

Advantageously, the process of the present invention makes it possible to efficiently separate methane from a gas stream comprising methane and ethylene at a relatively low pressure (e.g. at most 15 bar as mentioned above) and at a non-cryogenic temperature (e.g. of from 0 to 100° C. as mentioned above).

A further advantage of the process of the present invention is that all of the methane is recovered from the gas stream comprising methane and ethylene by means of the combination of sorption and rinse steps. If the rinse step would not be performed, the desorption step would result in a gas stream comprising ethylene and methane. In such a case, a distillation column would be needed to recover further methane from the gas stream resulting from the desorption step. Thus, the rinse step of the process of the present invention makes it possible that no distillation column is needed to recover further methane from the gas stream resulting from the desorption step. That is, advantageously, no “demethanizer” column is needed in the process of the present invention to separate methane from ethylene, by means of cryogenic distillation under high pressure. Thus, less energy is needed for compression of gas streams. Further, no energy is needed at all for refrigeration of a gas stream comprising methane and ethylene, before distillation in any “demethanizer” column, since all of the methane has already been separated in the sorption and rinse steps of the process of the present invention.

As is demonstrated in the present Examples, it has surprisingly appeared that advantageously the energy demand for the process of the present invention, especially the demand for compression and refrigeration energy, is significantly lower as compared to a process not comprising the sorption, rinse and desorption steps of the process of the present invention, but only comprising a distillation step using a “demethanizer” column as outlined above, in which latter comparative process no methane is removed from the gas stream comprising methane and ethylene before cryogenic distillation. Thus, the present process is a process that enables the recovery of methane from a gas stream comprising methane and ethylene in a way that is technically feasible, efficient and affordable since the energy demand is surprisingly lower as compared to the prior art process.

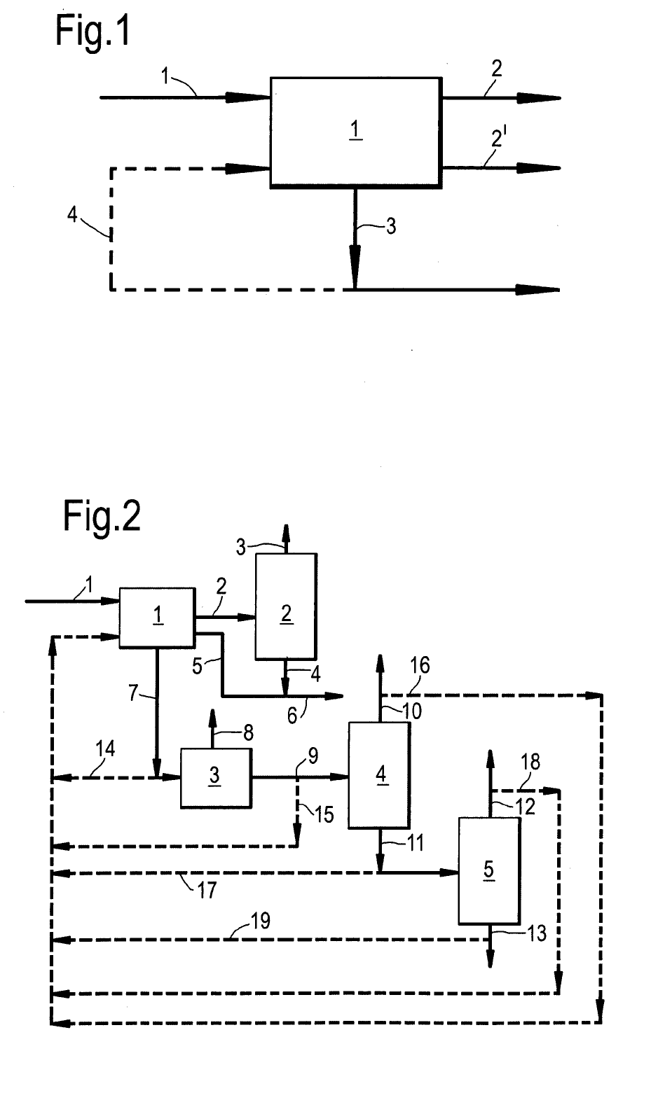

An example of an embodiment of the process of the present invention is schematically shown in FIG. 1. In said FIG. 1, a gas stream 1 comprising methane and ethylene is fed to sorption and desorption unit 1 which comprises a sorption agent which has a lower affinity for methane than for ethylene. The pressure of gas stream 1 is relatively high, for example in the range of from 5 to 15 bar, such that ethylene and part of the methane are sorbed by the sorption agent. A gas stream 2 comprising methane leaves sorption and desorption unit 1, which methane is not sorbed by the sorption agent in sorption and desorption unit 1.

After some time, the feed of gas stream 1 to sorption and desorption unit 1 is stopped. A gas stream comprising a compound for which the sorption agent has a higher affinity than for methane, which latter compound may for example comprise ethylene, ethane or a mixture of ethylene and ethane, is then fed to sorption and desorption unit 1. During this rinse step, the pressure of the latter gas stream is relatively high, for example in the range of from 5 to 15 bar, such that said compound for which the sorption agent has a higher affinity than for methane displaces methane sorbed by the sorption agent. A gas stream 2′ comprising methane leaves sorption and desorption unit 1. In addition to methane, gas stream 2′ may also comprise a certain amount of the compound for which the sorption agent has a higher affinity than for methane which compound is fed to sorption and desorption unit 1 in this rinse step.

After some time, the feed of the gas stream comprising the compound for which the sorption agent has a higher affinity than for methane to sorption and desorption unit 1 is stopped and the pressure in said unit is reduced. For example, the pressure in sorption and desorption unit 1 may be reduced to a pressure in the range of from 0.1 to 3 bar in a case wherein during the sorption and rinse steps the pressure is in the range of from 5 to 15 bar, as exemplified above. Through such pressure reduction the ethylene and the compound for which the sorption agent has a higher affinity than for methane that are sorbed by the sorption agent become desorbed. A gas stream 3 comprising ethylene and the compound for which the sorption agent has a higher affinity than for methane, that are desorbed from the sorption agent, leaves sorption and desorption unit 1.

Once the desorption of ethylene and the compound for which the sorption agent has a higher affinity than for methane is completed, the feed of gas stream 1 to sorption and desorption unit 1 is resumed and the above procedure is repeated. During the above-mentioned rinse step, the gas stream to be fed to sorption and desorption unit 1 may be a gas stream that is split off from gas stream 3, such as gas stream 4.

Said streams 2 and 2′ comprising methane may advantageously be used (recycled) partially or completely in a process wherein methane is used as a starting material (for further conversion of the recovered methane), for example in the above-mentioned process of oxidative coupling of methane (OCM). Alternatively, in a case where in addition to methane, gas stream 2′ also comprises a certain amount of the compound for which the sorption agent has a higher affinity than for methane, which compound is fed to sorption and desorption unit 1 in the rinse step, gas stream 2′ may be recycled to said unit, directly or via gas stream 1.

Preferably, the gas stream comprising methane and ethylene that is subjected to the sorption step of the process of the present invention comprises substantially no water. It is also preferred that said gas stream comprising methane and ethylene comprises substantially no hydrogen sulfide.

Within the present specification, by “substantially no” in relation to the amount of a specific component in a gas stream, it is meant an amount which is at most 1,000, preferably at most 500, preferably at most 100, preferably at most 50, more preferably at most 30, more preferably at most 20, and most preferably at most 10 ppmw of the component in question, based on the amount (i.e. weight) of said gas stream.

Further, in an embodiment of the process of the present invention, the gas stream comprising methane and ethylene that is subjected to the sorption step of the process of the present invention additionally comprises components other than said methane and ethylene, such as hydrogen, optionally nitrogen, carbon monoxide, carbon dioxide, ethane and hydrocarbons having 3 or more carbon atoms.

Suitably, said hydrocarbons having 3 or more carbon atoms comprise saturated and unsaturated hydrocarbons having 3 or more carbon atoms, including propane, propylene, butane and butene, and optionally saturated and unsaturated hydrocarbons having 5 or more carbon atoms.

As mentioned above, the gas stream comprising methane and ethylene that is subjected to the sorption step of the process of the present invention may additionally comprise nitrogen. Nitrogen may for example be present in a case where the gas stream originates from an OCM (oxidative coupling of methane) process wherein air is used as oxidant rather than pure oxygen.

In the above-mentioned embodiment of the process of the present invention, wherein the gas stream additionally comprises hydrogen, optionally nitrogen, carbon monoxide, carbon dioxide, ethane and hydrocarbons having 3 or more carbon atoms, said process comprises:

a sorption step which comprises contacting the gas stream comprising methane, ethylene, hydrogen, optionally nitrogen, carbon monoxide, carbon dioxide, ethane and hydrocarbons having 3 or more carbon atoms with a sorption agent which has a lower affinity for methane, hydrogen, nitrogen and carbon monoxide than for carbon dioxide, ethane, ethylene and hydrocarbons having 3 or more carbon atoms, resulting in sorption of hydrocarbons having 3 or more carbon atoms, ethane, ethylene, carbon dioxide and part of the methane by the sorption agent and in a gas stream comprising hydrogen, optionally nitrogen, carbon monoxide and methane;

a rinse step which comprises contacting a gas stream, comprising a compound for which the sorption agent has a higher affinity than for methane, with the sorption agent containing sorbed carbon dioxide, methane, ethylene, ethane and hydrocarbons having 3 or more carbon atoms, resulting in sorption of the compound for which the sorption agent has a higher affinity than for methane by the sorption agent, in desorption of methane from the sorption agent and in a gas stream comprising methane; and

a desorption step which comprises desorbing sorbed carbon dioxide, ethylene, ethane and hydrocarbons having 3 or more carbon atoms and the sorbed compound for which the sorption agent has a higher affinity than for methane resulting in a gas stream comprising carbon dioxide, ethylene, ethane and hydrocarbons having 3 or more carbon atoms and the compound for which the sorption agent has a higher affinity than for methane.

Further, preferably, in the above-mentioned embodiment of the process of the present invention, wherein the gas stream additionally comprises components other than methane and ethylene, the gas stream that is subjected to the sorption step comprises 40 to 90 mol % of methane, 0.5 to 45 mol % of ethylene, 0.01 to 3 mol % of hydrogen, 0 to 80 mol % of nitrogen, 0.1 to 5 mol % of carbon monoxide, 5 to 25 mol % of carbon dioxide, 0.1 to 25 mol % of ethane and 0.5 to 20 mol % of hydrocarbons having 3 or more carbon atoms. Said relative amounts are based on the total amount of the gas stream.

The sorption agents, pressures, temperatures, rinse method and sorption-desorption method (e.g. PSA) as discussed above also apply to the above-mentioned embodiment of the process of the present invention, wherein the gas stream additionally comprises components other than methane and ethylene. For example, in this embodiment, preferably, the gas stream to be used during the above-mentioned rinse step is a gas stream that is split off from said gas stream comprising carbon dioxide, ethylene, ethane and hydrocarbons having 3 or more carbon atoms and the compound for which the sorption agent has a higher affinity than for methane, resulting from the desorption step. In that way, advantageously, no external gas stream is needed to perform the rinse step and a greater process efficiency and integration is thereby achieved. Said advantage also applies to other embodiments as discussed below, wherein a gas stream is split off from a gas stream which comprises a compound for which the sorption agent has a higher affinity than for methane.

Preferably, in said embodiment, the process of the present invention additionally comprises a distillation step which comprises distilling the gas stream comprising hydrogen, optionally nitrogen, carbon monoxide and methane resulting from the sorption step, said distillation step resulting in a top stream comprising hydrogen, optionally nitrogen and carbon monoxide and a bottom stream comprising methane. Preferably, in said distillation step, the gas stream is distilled at a pressure in the range of from 20 to 40 bar, preferably 23 to 35 bar, and a temperature in the range of from −170 to −70° C., preferably −150 to −90° C. In the present specification, such temperature in a distillation step means the overhead temperature which is the temperature in the condenser at the top of the distillation column.

In case, in said embodiment, the gas stream comprising hydrogen, optionally nitrogen, carbon monoxide and methane resulting from the sorption step additionally comprises carbon dioxide, said gas stream may be split into a substream that is recycled to the sorption step and a substream that is bled instead of subjecting said gas stream to the above-mentioned distillation step.

Further, preferably, in said embodiment, the process of the present invention additionally comprises a carbon dioxide removal step which comprises removing carbon dioxide from the gas stream comprising carbon dioxide, ethylene, ethane and hydrocarbons having 3 or more carbon atoms and the compound for which the sorption agent has a higher affinity than for methane resulting from the desorption step, resulting in a gas stream comprising ethylene, ethane and hydrocarbons having 3 or more carbon atoms and the compound for which the sorption agent has a higher affinity than for methane. In said carbon dioxide removal step, carbon dioxide may be removed by any known method, such as treatment with an amine and then with a caustic agent, such as an aqueous monoethanolamine (MEA) absorption system and aqueous NaOH, respectively, as already mentioned above in the introduction of this specification. Preferably, the gas stream to be used during the above-mentioned rinse step is a gas stream that is split off from said gas stream comprising ethylene, ethane and hydrocarbons having 3 or more carbon atoms and the compound for which the sorption agent has a higher affinity than for methane, resulting from the carbon dioxide removal step.

Further, in said embodiment, the process of the present invention may additionally comprise a distillation step which comprises distilling the gas stream comprising ethylene, ethane and hydrocarbons having 3 or more carbon atoms and the compound for which the sorption agent has a higher affinity than for methane, which compound comprises one or more compounds selected from the group consisting of ethylene, ethane and hydrocarbons having 3 or more carbon atoms, resulting from the carbon dioxide removal step, said distillation step resulting in a top stream comprising ethylene and a bottom stream comprising ethane and hydrocarbons having 3 or more carbon atoms. Preferably, in said distillation step, the gas stream is distilled at a pressure in the range of from 10 to 40 bar, preferably 13 to 35 bar, and a temperature in the range of from −60 to 40° C., preferably −40 to 20° C. Preferably, the gas stream to be used during the above-mentioned rinse step is a gas stream that is split off from said top stream or from said bottom stream.

Still further, in said embodiment, the process of the present invention may additionally comprise a distillation step which comprises distilling the above-mentioned bottom stream comprising ethane and hydrocarbons having 3 or more carbon atoms, said distillation step resulting in a top stream comprising ethane and a bottom stream comprising hydrocarbons having 3 or more carbon atoms. Preferably, in said distillation step, the gas stream is distilled at a pressure in the range of from 10 to 40 bar, preferably 13 to 35 bar, and a temperature in the range of from −60 to 40° C., preferably −40 to 20° C. Preferably, the gas stream to be used during the above-mentioned rinse step is a gas stream that is split off from said top stream or from said bottom stream.

In an alternative embodiment, the process of the present invention may additionally comprise a distillation step which comprises distilling the above-mentioned gas stream comprising ethylene, ethane and hydrocarbons having 3 or more carbon atoms and the compound for which the sorption agent has a higher affinity than for methane, which compound comprises one or more compounds selected from the group consisting of ethylene, ethane and hydrocarbons having 3 or more carbon atoms, resulting from the carbon dioxide removal step, said distillation step resulting in a top stream comprising ethylene and ethane and a bottom stream comprising hydrocarbons having 3 or more carbon atoms. Preferably, in said distillation step, the gas stream is distilled at a pressure in the range of from 10 to 40 bar, preferably 13 to 35 bar, and a temperature in the range of from −60 to 40° C., preferably −40 to 20° C. Preferably, the gas stream to be used during the above-mentioned rinse step is a gas stream that is split off from said top stream or from said bottom stream.

Further, preferably, in said alternative embodiment, the process of the present invention additionally comprises a distillation step which comprises distilling the above-mentioned top stream comprising ethylene and ethane, said distillation step resulting in a top stream comprising ethylene and a bottom stream comprising ethane. Preferably, in said distillation step, the top stream is distilled at a pressure in the range of from 10 to 40 bar, preferably 13 to 35 bar, and a temperature in the range of from −60 to 40° C., preferably −40 to 20° C. Preferably, the gas stream to be used during the above-mentioned rinse step is a gas stream that is split off from said top stream or from said bottom stream.

An example of said embodiment of the process of the present invention, wherein the gas stream that is subjected to the sorption step additionally comprises components other than methane and ethylene, namely hydrogen, carbon monoxide, carbon dioxide, ethane and hydrocarbons having 3 or more carbon atoms, is schematically shown in FIG. 2. Hereinafter, the combination of ethylene, ethane and hydrocarbons having 3 or more carbon atoms may also be referred to as hydrocarbons having 2 or more carbon atoms. In said FIG. 2, a gas stream 1 comprising hydrogen, carbon monoxide, carbon dioxide, methane, ethylene, ethane and hydrocarbons having 3 or more carbon atoms is fed to sorption and desorption unit 1 which comprises a sorption agent which has a lower affinity for hydrogen, carbon monoxide and methane than for carbon dioxide and hydrocarbons having 2 or more carbon atoms. The pressure of gas stream 1 is relatively high, for example in the range of from 5 to 15 bar, such that hydrocarbons having 2 or more carbon atoms, carbon dioxide and part of the methane are sorbed by the sorption agent. A gas stream 2 comprising hydrogen, carbon monoxide and methane leaves sorption and desorption unit 1, which hydrogen, carbon monoxide and methane are not sorbed by the sorption agent in sorption and desorption unit 1. Gas stream 2 is sent to distillation column 2.

In distillation column 2, gas stream 2 comprising hydrogen, carbon monoxide and methane is distilled under such pressure and temperature conditions, for example those as described above, that separation between on the one hand hydrogen and carbon monoxide and on the other hand methane is effected. That is, a top stream 3 comprising hydrogen and carbon monoxide and a bottom stream 4 comprising methane leave distillation column 2. In case gas stream 2 additionally comprises carbon dioxide, gas stream 2 may be split into a substream that is sent (recycled) to sorption and desorption unit 1 and a substream that is bled (not shown in FIG. 2) instead of sending gas stream 2 to distillation column 2.

After some time, the feed of gas stream 1 to sorption and desorption unit 1 is stopped. A gas stream comprising a compound for which the sorption agent has a higher affinity than for methane, which compound comprises one or more compounds selected from the group consisting of ethylene, ethane and hydrocarbons having 3 or more carbon atoms and which compound may for example comprise ethylene, ethane or a mixture of ethylene and ethane, is then fed to sorption and desorption unit 1. During this rinse step, the pressure of the latter gas stream is relatively high, for example in the range of from 5 to 15 bar, such that said compound for which the sorption agent has a higher affinity than for methane displaces methane sorbed by the sorption agent. A gas stream 5 comprising methane leaves sorption and desorption unit 1. In addition to methane, gas stream 5 may also comprise a certain amount of the compound for which the sorption agent has a higher affinity than for methane which compound is fed to sorption and desorption unit 1 in this rinse step.

After some time, the feed of the gas stream comprising the compound for which the sorption agent has a higher affinity than for methane to sorption and desorption unit 1 is stopped and the pressure in said unit is reduced. For example, the pressure in sorption and desorption unit 1 may be reduced to a pressure in the range of from 0.1 to 3 bar in a case wherein during the sorption step the pressure is in the range of from 5 to 15 bar, as exemplified above. Through such pressure reduction carbon dioxide and hydrocarbons having 2 or more carbon atoms that are sorbed by the sorption agent become desorbed. A gas stream 7 comprising carbon dioxide and hydrocarbons having 2 or more carbon atoms, that are desorbed from the sorption agent, leaves sorption and desorption unit 1 and is sent to carbon dioxide removal unit 3.

Once the desorption is completed, the feed of gas stream 1 to sorption and desorption unit 1 is resumed and the above procedure is repeated.

In carbon dioxide removal unit 3, carbon dioxide is removed, via stream 8, from gas stream 7 comprising carbon dioxide and hydrocarbons having 2 or more carbon atoms, in a way as exemplified above. A gas stream 9 comprising hydrocarbons having 2 or more carbon atoms leaves carbon dioxide removal unit 3 and is sent to distillation column 4.

In distillation column 4, gas stream 9 comprising hydrocarbons having 2 or more carbon atoms is distilled under such pressure and temperature conditions, for example those as described above, that separation between on the one hand ethylene and on the other hand ethane and hydrocarbons having 3 or more carbon atoms is effected. That is, a top stream 10 comprising ethylene and a bottom stream 11 comprising ethane and hydrocarbons having 3 or more carbon atoms leave distillation column 4. Bottom stream 11 is sent to distillation column 5.

In distillation column 5, bottom stream 11 comprising ethane and hydrocarbons having 3 or more carbon atoms is distilled under such pressure and temperature conditions, for example those as described above, that separation between on the one hand ethane and on the other hand hydrocarbons having 3 or more carbon atoms is effected. That is, a top stream 12 comprising ethane and a bottom stream 13 comprising hydrocarbons having 3 or more carbon atoms leave distillation column 5.

As already mentioned above, once the desorption is completed, the feed of gas stream 1 to sorption and desorption unit 1 is resumed and the above procedure is repeated. During the above-mentioned rinse step, the gas stream to be fed to sorption and desorption unit 1 may be one or more of the following gas streams (as indicated by dashed lines in FIG. 2):

(1) a gas stream that is split off from gas stream 7 comprising carbon dioxide and hydrocarbons having 2 or more carbon atoms, such as gas stream 14;

(2) a gas stream that is split off from gas stream 9 comprising hydrocarbons having 2 or more carbon atoms, such as gas stream 15;

(3) a gas stream that is split off from top stream 10 comprising ethylene, such as gas stream 16;

(4) a gas stream that is split off from bottom stream 11 comprising ethane and hydrocarbons having 3 or more carbon atoms, such as gas stream 17;

(5) a gas stream that is split off from top stream 12 comprising ethane, such as gas stream 18; and

(6) a gas stream that is split off from bottom stream 13 comprising hydrocarbons having 3 or more carbon atoms, such as gas stream 19.

As already mentioned above, during the rinse step in the example illustrated in FIG. 2, the pressure of the gas stream fed to sorption and desorption unit 1 may for example be of from 5 to 15 bar. In such case, the pressure of gas streams 16 to 19 may have to be decreased whereas the pressure of gas streams 14 and 15 may have to be increased, before being fed to sorption and desorption unit 1.

Finally, bottom stream 4 comprising methane is combined with stream 5 comprising methane resulting in a single stream 6 comprising recovered methane. Said stream 4, stream 5 and/or stream 6, all comprising methane, may advantageously be used (recycled) partially or completely in a process wherein methane is used as a starting material (for further conversion of the recovered methane), for example in the above-mentioned process of oxidative coupling of methane (OCM). Alternatively, in a case where in addition to methane, gas stream 5 also comprises a certain amount of the compound for which the sorption agent has a higher affinity than for methane, which compound is fed to sorption and desorption unit 1 in the rinse step, gas stream 5 may be recycled to said unit, directly or via gas stream 1.

The invention is further illustrated by the following Examples.

### EXAMPLES AND COMPARATIVE EXAMPLE

In the Examples exemplifying the invention, the set-up as shown in FIG. 1 is used to recover methane from a gas stream comprising methane and ethylene, said set-up comprising sorption and desorption unit 1, as described in the description preceding these Examples.

In the Comparative Example exemplifying a prior art process, a distillation column (and not sorption and desorption unit 1 as shown in FIG. 1) is used to recover methane from a gas stream comprising methane and ethylene.

In the Examples, a gas stream 1 comprising 84.4 wt. % of methane and 15.6 wt. % of ethylene is fed at a temperature of 43° C. and a pressure of 9.8 bar to sorption and desorption unit 1 which comprises a sorption agent which has a lower affinity for methane than for ethylene. The ethylene and part of the methane from gas stream 1 are sorbed by the sorption agent. A gas stream 2 comprising 99.9+ wt. % of methane leaves sorption and desorption unit 1, which methane is not sorbed by the sorption agent in sorption and desorption unit 1.

The percentage of methane which leaves sorption and desorption unit 1 via gas stream 2 (which methane is not sorbed) is called the “methane rejection” and is based on the amount of methane as fed to sorption and desorption unit 1 via gas stream 1, and is 60, 75 or 90%, respectively, in the “PSA60”, “PSA75” and “PSA90” cases, respectively. Gas stream 2 is produced at the same temperature and pressure as gas stream 1 is fed to sorption and desorption unit 1, that is to say 43° C. and 9.8 bar, respectively.

After some time, the feed of gas stream 1 to sorption and desorption unit 1 is stopped. Further, a fraction of gas stream 3 comprising ethylene, said gas stream 3 resulting from the below-mentioned desorption step, is split into two substreams. One of these 2 substreams, namely gas stream 4, is compressed to 9.8 bar and is then fed to sorption and desorption unit 1 at a temperature of 43° C. and said same pressure. 7.4%, 4.6% and 1.8%, respectively, of the desorbed ethylene is diverted from gas stream 3 to gas stream 4, in the “PSA60”, “PSA75” and “PSA90” cases, respectively. The ethylene from gas stream 4 is sorbed by the sorption agent and sorbed methane is desorbed from the sorption agent in sorption and desorption unit 1. A gas stream 2′ comprising 99.0+ wt. % of methane leaves sorption and desorption unit 1, which methane is desorbed from the sorption agent in sorption and desorption unit 1. Gas stream 2′ is produced at the same temperature and pressure as gas stream 4 is fed to sorption and desorption unit 1, that is to say 43° C. and 9.8 bar, respectively.

After some time, the feed of gas stream 4 to sorption and desorption unit 1 is stopped and the pressure in said unit is reduced from 9.8 bar to 1 bar, thereby inducing the desorption step of the process of the present invention. The sorbed ethylene subsequently becomes desorbed from the sorption agent and leaves sorption and desorption unit 1 via gas stream 3, comprising 99.9+ wt. % of ethylene, at a temperature of 23° C. and a pressure of 1 bar.

As already mentioned above, in the Comparative Example (“base case”), a distillation column (and not sorption and desorption unit 1 as shown in FIG. 1) is used to recover methane from a gas stream comprising methane and ethylene. Said gas stream is the same as gas stream 1, that is to say a gas stream comprising 84.4 wt. % of methane and 15.6 wt. % of ethylene. Said gas stream is compressed to 9.8 bar in a first compressor, further compressed to 32.9 bar in a second compressor and finally cooled to −84° C. before it enters the distillation column which is a column having 36 theoretical stages. In the distillation column, the following 2 streams are separated: a top stream comprising 99.9+ wt. % of methane at an (overhead) temperature of −98° C. and a pressure of 31.1 bar and a bottom stream comprising 99.8+ wt. % of ethylene at a temperature of −5° C. and a pressure of 31.3 bar.

In relation to the distillation column as used in this base case, the reflux ratio and the distillate-to-feed ratio are 0.85 and 0.89, respectively. By said “reflux ratio”, reference is made to the molar ratio of the molar flow rate of the “reflux stream”, which is that part of the stream that leaves the condenser at the top of the distillation column which is sent back to that column, divided by the molar flow rate of the “distillate”, which is that part of the stream that leaves the condenser at the top of the distillation column which is not sent back to that column. By said “distillate-to-feed ratio”, reference is made to the molar ratio of the molar flow rate of said “distillate” divided by the molar flow rate of the feedstream that is fed to that column (the “feed”).

In Table 1 below, the compression and refrigeration energy needed to recover methane from the gas stream comprising methane and ethylene is included for all of the above-discussed cases, that is to say both the comparative “base case” (Comparative Example) and the cases of the invention which are the “PSA60”, “PSA75” and “PSA90” cases (Examples). Said energy is expressed as kilowatt hour (“kWh”; 1 kWh=3.6 megajoules) per kilogram (kg) of ethylene.

TABLE 1

| | | kWh/kg of |
|---|---|---|
| Case | Configuration | ethylene |
| base case | distillation (comparative) | 1.77 |
| PSA60 | PSA including rinse step | 1.42 |
| PSA75 | PSA including rinse step | 1.22 |
| PSA90 | PSA including rinse step | 0.99 |

From Table 1 above, it surprisingly appears that the energy needed to recover methane from the gas stream comprising methane and ethylene is advantageously lowest in case the process of the present invention is carried out. That is, in all of the “PSA60”, “PSA75” and “PSA90” cases which exemplify the process of the present invention, the energy needed to recover said methane, is advantageously lower than the energy needed to effect the same in the “base case” in which latter case such process is not applied but a distillation step is performed.

Thus, surprisingly, this advantageous different energy effect, as compared to the prior art process wherein a distillation step is performed, is obtained with the process of the present invention even though in the present invention the sorption and desorption process includes an intermediate rinse step which latter step requires a relatively high pressure, as demonstrated in the “PSA60”, “PSA75” and “PSA90” cases.

1. A process for recovering methane from a gas stream comprising methane and ethylene, comprising:
a sorption step which comprises contacting the gas stream comprising methane and ethylene with a sorption agent which has a lower affinity for methane than for ethylene, resulting in sorption of ethylene and part of the methane by the sorption agent and in a gas stream comprising methane;
a rinse step which comprises contacting a gas stream, comprising a compound for which the sorption agent has a higher affinity than for methane, with the sorption agent containing sorbed ethylene and methane, resulting in sorption of the compound for which the sorption agent has a higher affinity than for methane by the sorption agent, in desorption of methane from the sorption agent and in a gas stream comprising methane; and
a desorption step which comprises desorbing sorbed ethylene and the sorbed compound for which the sorption agent has a higher affinity than for methane resulting in a gas stream comprising ethylene and the compound for which the sorption agent has a higher affinity than for methane.

2. The process according to claim 1, wherein the gas stream that is subjected to the sorption step comprises 50 to 99 mol % of methane and 1 to 50 mol % of ethylene.

3. The process according to claim 1, wherein the compound for which the sorption agent has a higher affinity than for methane comprises one or more compounds selected from the group consisting of ethylene, ethane and hydrocarbons having 3 or more carbon atoms.

4. The process according to claim 3, wherein the gas stream comprising a compound for which the sorption agent has a higher affinity than for methane comprises ethylene, ethane or a mixture of ethylene and ethane, preferably ethylene.

5. The process according to claim 1, wherein desorption in the desorption step is effected by reducing the pressure.

6. The process according to claim 5, wherein the pressure in the sorption and rinse steps is in the range of from 5 to 15 bar, and the pressure in the desorption step is in the range of from 0.1 to 3 bar.

7. A process according to claim 1, wherein the gas stream comprising methane and ethylene that is subjected to the sorption step additionally comprises hydrogen, nitrogen, carbon monoxide, carbon dioxide, ethane and hydrocarbons having 3 or more carbon atoms, which process comprises:
a sorption step which comprises contacting the gas stream comprising methane, ethylene, hydrogen, nitrogen, carbon monoxide, carbon dioxide, ethane and hydrocarbons having 3 or more carbon atoms with a sorption agent which has a lower affinity for methane, hydrogen, nitrogen and carbon monoxide than for carbon dioxide, ethane, ethylene and hydrocarbons having 3 or more carbon atoms, resulting in sorption of hydrocarbons having 3 or more carbon atoms, ethane, ethylene, carbon dioxide and part of the methane by the sorption agent and in a gas stream comprising hydrogen, nitrogen, carbon monoxide and methane;
a rinse step which comprises contacting a gas stream, comprising a compound for which the sorption agent has a higher affinity than for methane, with the sorption agent containing sorbed carbon dioxide, methane, ethylene, ethane and hydrocarbons having 3 or more carbon atoms, resulting in sorption of the compound for which the sorption agent has a higher affinity than for methane by the sorption agent, in desorption of methane from the sorption agent and in a gas stream comprising methane; and
a desorption step which comprises desorbing sorbed carbon dioxide, ethylene, ethane and hydrocarbons having 3 or more carbon atoms and the sorbed compound for which the sorption agent has a higher affinity than for methane resulting in a gas stream comprising carbon dioxide, ethylene, ethane and hydrocarbons having 3 or more carbon atoms and the compound for which the sorption agent has a higher affinity than for methane.

8. The process according to claim 7, wherein the gas stream that is subjected to the sorption step comprises 40 to 90 mol % of methane, 0.5 to 45 mol % of ethylene, 0.01 to 3 mol % of hydrogen, 0 to 80 mol % of nitrogen, 0.1 to 5 mol % of carbon monoxide, 5 to 25 mol % of carbon dioxide, 0.1 to 25 mol % of ethane and 0.5 to 20 mol % of hydrocarbons having 3 or more carbon atoms.

9. The process according to claim 7, additionally comprising a distillation step which comprises distilling the gas stream comprising hydrogen, optionally nitrogen, carbon monoxide and methane resulting from the sorption step, said distillation step resulting in a top stream comprising hydrogen, optionally nitrogen and carbon monoxide and a bottom stream comprising methane.

10. The process according to claim 7, additionally comprising a carbon dioxide removal step which comprises removing carbon dioxide from the gas stream comprising carbon dioxide, ethylene, ethane and hydrocarbons having 3 or more carbon atoms and the compound for which the sorption agent has a higher affinity than for methane resulting from the desorption step, resulting in a gas stream comprising ethylene, ethane and hydrocarbons having 3 or more carbon atoms and the compound for which the sorption agent has a higher affinity than for methane.

11. The process according to claim 10, additionally comprising a distillation step which comprises distilling the gas stream comprising ethylene, ethane and hydrocarbons having 3 or more carbon atoms and the compound for which the sorption agent has a higher affinity than for methane, which compound comprises one or more compounds selected from the group consisting of ethylene, ethane and hydrocarbons having 3 or more carbon atoms, resulting from the carbon dioxide removal step, said distillation step resulting in a top stream comprising ethylene and a bottom stream comprising ethane and hydrocarbons having 3 or more carbon atoms.
